(19) United States
(12) Patent Application Publication
(10) Pub. No.: US 2024/0013898 A1
(43) Pub. Date: Jan. 11, 2024

(54) SYSTEM AND METHOD FOR FACILITATING TIMELY PROPHYLACTIC COLORECTAL CANCER EVALUATIONS

(71) Applicant: Arvind Ravi, Cambridge, MA (US)

(72) Inventor: Arvind Ravi, Cambridge, MA (US)

(21) Appl. No.: 18/350,711

(22) Filed: Jul. 11, 2023

Related U.S. Application Data
(60) Provisional application No. 63/368,084, filed on Jul. 11, 2022.

(51) Int. Cl. G16H 40/20; G16H 50/70; G16H 10/60; G06F 40/40; G06F 40/103

(52) U.S. Cl. CPC G16H 40/20; G16H 50/70; G16H 10/60; G06F 40/40; G06F 40/103

(57) ABSTRACT

A system for early detection of colorectal cancer by facilitating timely prophylactic colonoscopies and screenings is disclosed. The system comprises three parts: a computer memory for storing aggregated electronic health records from a multitude of patients including size and history of polyps and cancers identified during previous screenings, laboratory values, vital signs, and medical notes and trained machine learning models; a computer or network of computers running these models; and an electronic device. The computer can convert aggregated electronic health records into a single standardized data structure format and further executes a priority score model configured to predict a priority score that indicate a patient's need for urgent re-screening based on an input electronic health record of a patient having the standardized data structure format. The electronic device is configured with a healthcare provider-facing interface that can display the predicted one or more overdue patients and their priority scores.

### CROSS REFERENCE AND PRIORITY

This application claims the benefit of U.S. Provisional Application No. 63/368,084, filed Jul. 11, 2022, which is incorporated herein by reference in its entirety for all purposes.

### TECHNICAL FIELD

The present disclosure is generally related to the field of treatment and prophylaxis. More particularly, the present disclosure is related to methods and systems that incorporate natural language processing (NLP) and artificial intelligence/machine learning (AI/ML) technology for improved prophylaxis of colorectal cancer.

### BACKGROUND OF THE INVENTION

### Early Detection of Cancer

Apart from skin cancers, colorectal cancer ranks third in terms of prevalence among all cancers for both men and women in the United States. Essentially, colorectal cancer is a condition where cells in the colon or rectum proliferate uncontrollably. Occasionally, unusual growths known as polyps may develop in these regions. As time progresses, there's a possibility that some of these polyps may transform into cancer. According to the American Cancer Society, around 150,000 new cases of colorectal cancer were reported in the United States in 2021.

The likelihood of developing colorectal cancer escalates with advancing age. Other risk indicators encompass conditions like inflammatory bowel disease, personal or family history of colorectal cancer or colorectal polyps, and genetic syndromes like familial adenomatous polyposis or hereditary non-polyposis colorectal cancer. Certain lifestyle choices can also heighten the risk. These include inadequate regular physical exercise, a diet deficient in fruits and vegetables, consumption of low-fiber and high-fat foods, obesity, alcohol intake, and tobacco use.

The prognosis for colorectal cancer patients markedly improves if surgery and other treatments are initiated before the cancer spreads, or metastasizes. Therefore, routine screening for colorectal cancer is the most effective way to lower one's risk of developing this disease and its complications. It's important to note that nearly all colorectal cancers start as precancerous polyps, or abnormal growths, in the colon or rectum. These polyps can exist for years before evolving into invasive cancer and might not produce noticeable symptoms, particularly in the early stages. Thus, regular colorectal cancer screenings provide a powerful tool to detect and remove these precancerous polyps before they become cancerous, preventing colorectal cancer and its potential complications. Moreover, these screenings can identify colorectal cancer at an early stage, when treatment has the highest chance of success. As per the American Cancer Society, the five-year survival rate for patients with early-stage or localized colon cancer is 91%. However, for those with metastatic or advanced disease, the survival rate drops significantly to just 14%.

The current practice for scheduling patients for screening or surveillance colonoscopies primarily involves a conversation with their primary care providers, or a reminder letter from their gastroenterologist indicating their next visit. Regrettably, this process has some deficiencies, with roughly 30% of patients aged between 50 and 75 not receiving timely screenings, according to the Centers for Disease Control. Reported reasons for this neglect include a lack of patient understanding of the necessity for screening and insufficient time during office visits for detailed discussions with their healthcare provider.

Some more advanced scheduling methods have been proposed, such as patient registries and reminder systems aimed at both patients and healthcare providers. However, even when these systems are used appropriately, they result in only slight increases in screening rates. Importantly, these advanced methods do not easily allow for prioritizing patients based on risk, which would enable a more effective allocation of screening resources.

It would therefore be beneficial to enhance the precision and uniformity of colorectal cancer screening in areas where it is frequently conducted. In doing so, we could create tools and technologies that could potentially improve or promote preventive cancer screening in regions where it is less commonly performed.

### Artificial Intelligence/Machine Learning and Natural Language Processing Systems

Artificial Intelligence (AI) and Machine Learning (ML) systems can be extremely valuable for processing and analyzing information, potentially aiding medical professionals in their decision-making process. For instance, these systems can include diagnostic decision-support tools that utilize clinical decision algorithms, rules, decision trees, or other mechanisms. Such tools may assist physicians in making accurate diagnoses.

While decision-making systems have been developed, they are not extensively implemented in medical practices due to certain limitations that impede their integration into the daily operations of healthcare organizations. For instance, a patient might be attended by numerous healthcare professionals across various settings. This results in the patient's data being dispersed across multiple computer systems in both structured and unstructured formats. Additionally, these systems typically don't focus on disease treatment or prevention, and they often fail to assist clinicians in determining the best course of action for patient care.

Natural Language Processing (NLP) broadly refers to a software's ability to automatically handle natural language, such as spoken or written text. NLP systems can be set up to carry out tasks like Optical Character Recognition (OCR), which involves converting images of typed, handwritten, or printed text from scanned documents, photos of documents, or scene-photos into text that machines can process. Moreover, NLP systems can execute feature extraction, where they pull out feature representations suitable for particular NLP tasks and AI/ML models from the input text. Considering estimates suggesting that as many as three-quarters of medical communications happen via fax, the capacity to efficiently digitize and process large volumes of unstructured and image-based data could render these formats more accessible to advanced AI/ML systems.

Current methods used by hospitals to track colorectal cancer screening and follow-ups vary widely. Traditionally, doctors have communicated when the next screening should occur in a conversation after the procedure, or shortly after via a phone call or a mailed letter. Unfortunately, this approach largely leaves the responsibility on the patients to remember this crucial medical guidance. Other tracking systems in use range from physician-centered methods such as notecards and spreadsheets to more advanced systems where recall data is integrated into the clinical record (for example, via a Healthcare Maintenance field) or a clinical registry.

However, even more advanced systems are limited in their scope, as they can only operate based on the minimal clinical details recorded in the relevant structured fields. They often lack detailed descriptions of the pathologic findings (like polyp size, number, histology, etc.), or any linkage to laboratory data that could be used to provide more sophisticated risk assessments for these patient populations.

Clinical evaluations, either through a clinic visit to assess symptoms or a screening procedure like a colonoscopy, can offer valuable insights into a patient's underlying risk of colon cancer, based on factors like family history of cancer or detection of precancerous lesions. However, objective measurements such as vital signs and lab data may provide further understanding of risk, particularly because these can be performed outside of regular screening and surveillance visits, thereby enhancing the opportunity to gather relevant data over time. Furthermore, relying solely on colonoscopies has limitations due to variations in the quality of preparation, patient anatomy, and provider expertise, which can result in less than perfect detection of underlying colonic lesions.

It would therefore be desirable to provide methods and systems which incorporate AI/ML and NLP technology to aid in the early detection of colorectal cancer, especially in regard to facilitating timely prophylactic screening for high-risk patients.

### BRIEF SUMMARY OF INVENTION

As an overview and summary, one aspect of this disclosure is directed to a system and method for improved prophylaxis of colorectal cancer by leveraging natural language processing and machine learning to aid in the early detection of colorectal cancer, especially in regard to facilitating timely prophylactic colonoscopies and screenings. The overall purpose of the disclosed system and method is to allow healthcare providers to identify high-risk patients requiring urgent re-screening while still enabling said healthcare providers to reasonably schedule patients in a timelike fashion considering resource limitations. The system includes three components:

The first component is a computer memory, such as a large data storage device or devices, which stores comprehensive electronic health records from numerous patients of varying ages, health statuses, and demographics. These records include, but are not limited to, the size and history of polyps and cancers identified during previous screenings, laboratory values, vital signs, and medical notes. These accumulated health records are sourced from one or more systems and are organized in different data structures than those used in existing or legacy systems. Furthermore, these records may be converted into a unified, standardized data structure format and are preferably arranged in an ordered format, such as chronological order. In addition to these health records, the computer memory also stores trained machine learning models to be used at various stages of the system's operation.

The second component is a computer, which could be a single computer or a network of computers or processing units sharing a processing task, that runs one or more trained machine learning models. The specific training and purposes of these models will be discussed further in the specification.

The third component is an electronic device used by a healthcare provider to determine whether a patient requires urgent re-screening within the next three months. This could be a computer terminal or workstation, tablet, smartphone, or any other type of computing device equipped with a screen display. The screen is configured with a healthcare provider-facing interface displaying a list of one or more patients and their respective recall priority scores, which indicate whether the patients require urgent re-screening for colorectal cancer. In the preferred embodiment, the recall priority score for each patient ranges from 1 to 10. A score exceeding 7 signifies the need for an urgent and comprehensive prophylactic colonoscopy or screening/surveillance exam within the next three months.

### BRIEF DESCRIPTION OF THE DRAWINGS

The invention will be readily understood by the following detailed description in conjunction with the accompanying drawings, wherein like reference numerals designate like structural elements, and in which:

FIG. 1 is a schematic diagram of the overall system including aggregated electronic health records, a computer executing trained deep learning models, and an electronic device used by a healthcare provider configured to allow the healthcare provider to interact with the system, receiving predictions from the deep learning models for the healthcare provider selected patients and having an interface to present such information on the electronic device's display.

FIG. 2 is a flow diagram illustrating a method for facilitating timely prophylactic colorectal cancer evaluations based on aggregated electronic health records, in accordance with one embodiment of the present invention.

The drawings described herein are for illustration purposes and are not intended to limit the scope of the present subject matter in any way.

### DETAILED DESCRIPTION

In the following description, to better understand the aforementioned purposes, features, and advantages of the present invention, numerous specific details are set forth in order to provide a thorough understanding of the claimed subject matter. It should be noted that these details and examples are provided to merely aid in understanding the descriptions, and they do not, in any way, limit the scope of the present invention. The present invention can also be implemented in other modes different from those described herein and the present invention is not limited to the specific embodiments disclosed below.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The specification may refer to “an”, “one” or “some” embodiment(s) in several locations. This does not necessarily imply that each such reference is to the same embodiment(s), or that the feature only applies to a single embodiment. A single feature of different embodiments may also be combined to provide other embodiments.

Furthermore, as used herein, the singular forms “a”, an” and “the” are intended to include the plural forms as well, unless expressly stated otherwise, It will be further understood that the terms “includes”, comprises“, including” and/or “comprising” when used in this specification, specify the presence of stated features, integer steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

The computer-readable storage medium (“memory”) can be a tangible device that can retain and store instructions for use by an instruction execution device. The computer-readable storage medium may be, without limitation, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including, but not limited to, an object-oriented programming language such as Python, Java, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus, or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### System Overview

This disclosure describes a new system and method for improved prophylaxis of colorectal cancer by leveraging natural language processing and machine learning to aid in the early detection of colorectal cancer, especially in regard to facilitating timely prophylactic colonoscopies and screenings. The main innovation of the disclosed system and method resides in the distinctive combination and utilization of various Natural Language Processing and Machine Learning modules. These are specifically designed to facilitate timely preventive colonoscopies and screenings while considering the resource limitations of healthcare providers. Moreover, a new approach to colorectal cancer prevention is disclosed. Specifically, it introduces a preventive treatment plan where a qualified medical practitioner performs a colonoscopy or screening in response to a generated priority score.

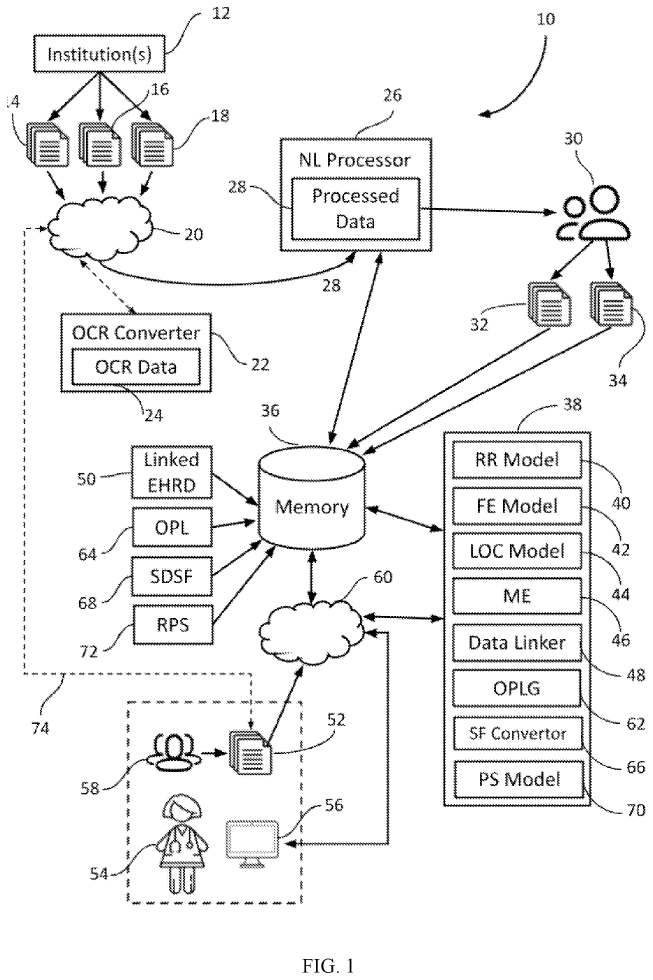

FIG. 1 illustrates a system 10 for facilitating timely prophylactic colonoscopies and screenings. The system includes three primary components:

First, there is described a computer memory 36, e.g., mass data storage device or devices, storing, among other things, post-NLP processed data 28, a patient recall labeled dataset 32 created by subject matter expert(s) 30, a polyps/cancer features labeled dataset 34 created by subject matter expert(s) 30, a linked electronic health records database (LEHRD) 50, an overdue patients list (OPL) 64, standard format data (SDSF) 68, a recall priority sore (RPS) 72, and electronic health records (EHRs) 52 collected during the normal course of operations by a healthcare provider 54 from a multitude of patients 58 of diverse age, health conditions, and demographics, the records including, but not limited to, procedure reports, clinical notes, pathology reports, and laboratory values from clinical databases for said patients. The EHRs 52 are obtained from one or more sources and may be organized in different data structure types than those used in said current systems or legacy systems.

Secondly, the system 10 includes a computer 22 configured to perform optical character recognition on scanned procedure reports 14, clinical notes 16, and pathology reports 18 obtained from large numbers of patients from different institutions 12 (e.g., hospital systems, university medical centers, clinics) as necessary. Herein, the term “computer” is intended to refer to a single computer or a system of computers or processing units sharing a processing task, together with ancillary memory. Here, ancillary memory is memory that is, for example, instantiated for local use by a networking subsystem. The ancillary memory is, for example, closely coupled to peripheral processors such as WAN, LAN, Bluetooth, and other such peripheral controllers or devices on the same substrate. The procedure reports 14, clinical notes 16, and pathology reports 18 are transmitted over a computer network 20 to the computer 22 where they undergo optical character recognition, resulting in generated recognized textual data 24.

The system 10 further includes a computer 26 configured to perform natural language processing on textual data. Textual data (or the recognized textual data if optical character recognition is first performed) is transmitted over the computer network 20 to the NLP computer system 26 where it undergoes natural language processing resulting in generated processed data 28 containing relevant extracted data. In the preferred embodiment, the generated processed data 28 undergoes data harmonization based on the category of the obtained data. For example, time-based data is standardized by assigning a numerical time interval (e.g., ‘2’) along with its associated unit of time (e.g., ‘years’). Moreover, data values that fall outside a reasonable recall range (e.g., intervals exceeding 10 years) are excluded from the dataset. Furthermore, the data is integrated to ensure that only a single recall interval exists from a procedure report. In cases where multiple recall intervals are identified, the most recent one is utilized, as it is highly likely to correspond to the recommendations section of the procedure report. The processed data 28 are stored in memory 36.

The processed data 28 originating from the procedure reports 14 and the clinical notes 16 are used by subject matter expert(s) 30 to create the patient recall labeled dataset 32. The processed data originating from the procedure reports 14 and the pathology reports 18 are used by subject matter expert(s) 30 to create the polyps/cancer features labeled dataset 34.

Similarly, as indicated by the dashed line 74, the healthcare provider's EHRs 52 could be processed by computers 22 and 26 if necessary. Once processed, they are stored in the memory 36. Such optical character recognition by the computer 22 and natural language processing by the computer 26 transform the EHRs 52 into data formats usable as inputs to the RRM 40, the FEM 42, and the metadata extractor 46 (described below).

The system 10 further includes a computer 38 executing one or more deep learning models 40, 42, 44. Herein, deep learning models refer to the use of artificial neutral networks with several layers to model and understand complex patterns and relationships in data. Deep learning model types or architectures that are executed by the computer 38 include, but are not limited to, Feedforward Neural Networks (FNNs), Convolutional Neural Networks (CNNs), and Recurrent Neural Networks (RNNs).

The recommended recall deep learning model (“RRM”) 40 is trained on the patient recall labeled dataset 32 and is configured to accept processed data 28 corresponding to input procedure reports 14 and clinical notes 16 to generate and output a recommended recall interval (e.g., 3 months) corresponding to the time interval from a given date during which the patient should receive a prophylactic colorectal cancer evaluation. In the preferred embodiment, the RRM 40 is a Named Entity Recognition model.

The feature extraction deep learning model (“FEM”) 42 is trained on the polyps/cancer features labeled dataset 34 and configured to accept processed data 28 corresponding to input procedure reports 14 and pathology reports 18 to generate and output polyps and/or cancer features.

The RRM 40 and FEM 42 are models which can be trained from scratch on a custom dataset or pretrained transformer models that then undergo fine-tuning (continued training) on a custom dataset (supervised clinical annotations).

The likelihood of cancer deep learning model (“LOCM”) 44 is a machine learning model which uses the output of the Recommended Recall Model 40, the output of the Feature Extraction Model 42, and various structured laboratory data taken from a healthcare provider's electronic health records 52 as input features in order to generate priors on the likelihood of a given patient having cancer for a given pathologic evaluation. In the preferred embodiment, the LOCM 44 is a Bayesian lasso model.

The computer 38 further executes a metadata extractor 46 and a data linker 48. The metadata extractor 46 uses heuristics-based regular expressions to extract relevant document metadata (e.g., date, patient identifier) from the processed data 28 stored in the memory 36 relating to the EHRs 52. The data linker 48 uses the extracted document metadata generated by the metadata extractor 46 to generate the database 50. Particularly, the data linker 48 generates a database that joins pointers to individual documents from the electronic health records 52 that correspond to the same visit for each patient. This database is referred to as the linked electronic health records database 50. In the preferred embodiment, initial joining is done on shared extracted document metadata. Afterward, additional heuristics are applied to select the highest likelihood match when one-to-many matching is present (e.g., selecting the most likely match from multiple procedure reports, clinical notes, and the like that are generated after a given procedure is performed).

The computer 38 additionally performs the execution of an overdue patient list generator (OPLG) 62, which is responsible for identifying patients who are overdue for a colonoscopy or screening from the selected pool of patients 58 recorded in the healthcare provider's electronic health records 52. The identified overdue patients are then stored in the memory 36 as an overdue patients list (OPL) 64. This process is accomplished through the following steps for each patient:

The OPLG 62 uses the linked database 50 to identify a singular patient's relevant information (e.g., procedure reports, clinical notes, pathology reports) from the stored processed data 28 associated with the pool of patients 58 recorded in the healthcare provider's electronic health records 52 and/or a specific set of patients chosen by the health care provider 54. The clinical notes and procedure reports from the identified information are then processed by the RRM 40 to generate a recommended recall interval. For example, the recommended recall interval could be three months or three years. Furthermore, the individual patient's procedure reports, clinical notes, and pathology reports are processed by the metadata extractor 46 to generate various data such as the time and date of the reports/notes as well as the last documented procedure date.

The OPLG 62 then compares the recommended recall interval generated by the RRM 40 to the last documented procedure date identified by the metadata extractor 46. Here, the recommended recall interval corresponding to the most recent procedure date as identified by the metadata extractor 46 defines a window from that last documented procedure date during which the patient should have completed a follow-up colonoscopy or screening. Patients for whom that time window has fully elapsed without receiving a follow-up colonoscopy or screening are designated as “overdue” and are added to the overdue patients list 64.

The computer 38 further executes a standard format converter (SFC) 66. For each patient on the OPL 64, the SFC 66 converts processed data 28 linked with said patient into a standardized data structure format 68. In the preferred embodiment, the standardized data structure format includes:
a) recall delta: time difference between analysis date (the date of when the system was utilized to evaluate data) and recall_final in years (the recall due date based on the recall interval reported in the most recent post-procedure letter to the patient if one is present). If no such post-procedure letter to the patient is present, then recall_final is based on the recall interval from the procedure report.
b) indications: one hot encoded versions of the following indications for recall based on the most recent procedure report/pathology report/post-procedure letter:
indication_surveillance;
indication_prior_adenoma;
indication_prior_high_risk_adenoma;
indication_personal_history_colon_cancer;
indication_EMR;
indication_post_EMR;
indication_family_history_polyps;
indication_family_history_of_colon_cancer;
indication_anemia;
indication_bleeding;
indication_poor_prep;
indication_ulcerative_colitis;
indication_crohns;
Indication_polyposis_syndrome; and
Indication_high_grade_dysplasia
c) clinical laboratory data associated with said patient including:
lab_sodium
lab_potassium
lab_chloride
lab_bicarbonate
lab_bun
lab_creatinine
lab_glucose
lab_ast
lab_alt
lab_albumin
lab_bilirubin
lab_alkaline_phosphate
lab_hct
lab_mcv
lab_rdw
lab_platelets
lab_wbc

The computer 38 further executes a priority score model 70. The priority score model 70 is trained on the processed data 28 relating to the reports and notes 14, 16, and 18, which have been converted by the SFC 66 into the standardized data structure format 68. Said input data represents a physician-identified risk of recall miss based on the features used for the Likelihood of Cancer Model 44 (i.e., the relative priority with which patients should be called back based on the likelihood and severity of underlying colon cancer or a precancerous lesion). The priority score model 70 outputs a recall priority score 72 between 1 and 10. A score over 7 indicates the need for an urgent and thorough colonoscopy or screening/surveillance exam within the next three months. This cutoff of 7 is set to identify roughly the highest 20th percentile of patients by risk while still enabling hospital systems to reasonably schedule patients in a timely fashion given resource limitations. This cutoff can be readily adjusted by the healthcare provider 54 as necessary if resource limitations change. Preferably, the cutoff is adjusted by the healthcare provider to provide the maximum number of colonoscopies and screenings/surveillance exams to the patients that require such services the most given the healthcare provider's resources.

Thirdly, the system includes an electronic device 56 for use by a healthcare provider treating the patient (e.g., computer terminal or workstation, tablet, smartphone, or other type of computing device having a screen display) which is configured with a healthcare provider-facing interface displaying the predicted recall priority score 72 and the pertinent past medical events of the of one or more patients 58 to a healthcare provider 54. The display of the recall priority score and relevant past medical events assist the healthcare provider 54 with identifying patients with the highest risk of future colorectal cancer and colorectal cancer complications. Such high-risk patients would then be contacted by the healthcare provider, scheduled to receive prophylactic colonoscopies and screenings, and receive timely prophylactic colonoscopies and screenings performed by the healthcare provider.

The precise physical location and implementation of the predictive models and related computer or computer system 38 may vary. In some instances, it may be physically located at a medical system or hospital serving affiliated facilities, primary care physician offices, related clinics, etc. In other situations, it may be centrally located and receive EHRs 52 and transmit predicted future clinical events and related prior medical events over wide area computer networks and service a multitude of unrelated healthcare institutions in a fee for service, subscription, standalone product, or other business models. In all situations, appropriate data security and HIPAA compliance procedures are in place.

### Method of Use Example

In the normal course of healthcare provision, a healthcare provider 54 provides medical services to a multitude of patients 58. The healthcare provider has access to a plurality of electronic health records 52 associated with the multitude of patients, the EHRs being collected during the normal course of operations by the healthcare provider or other medical institutions and including, without limitation, procedure reports, clinical notes, pathology reports, and clinical database records containing laboratory data. The EHRs are often organized in a variety of different data structures and may not necessarily be organized in a data structure used by the healthcare provider's current system.

A goal of the healthcare provider may be to prophylactically prevent the greatest amount of colorectal cancer and colorectal cancer complications for their patients 58. However, due to hospital resource limitations (e.g., limited time and effort of the hospital's employed medical professionals) and limitations in patient scheduling and tracking, some patients at a high risk of colorectal cancer do not receive the proper colonoscopies and screenings necessary to prevent future colorectal cancer development. A method of use of the disclosed system 10 by a healthcare provider (e.g. nurses, primary care physicians, specialized doctors) to overcome the aforementioned real-world hospital environment limitations for improved prophylaxis of colorectal cancer follows.

Ideally, before employing the described system, the deep learning models 40, 42, 44 housed within the computer 38 are initially trained on a collection of procedure reports 14, clinical notes 16, and pathology reports 18 that have undergone scanning and preprocessing by the OCL Converter 22 and the NL Processor 26, respectively. If needed, these deep learning models can be further refined and adjusted for optimal performance.

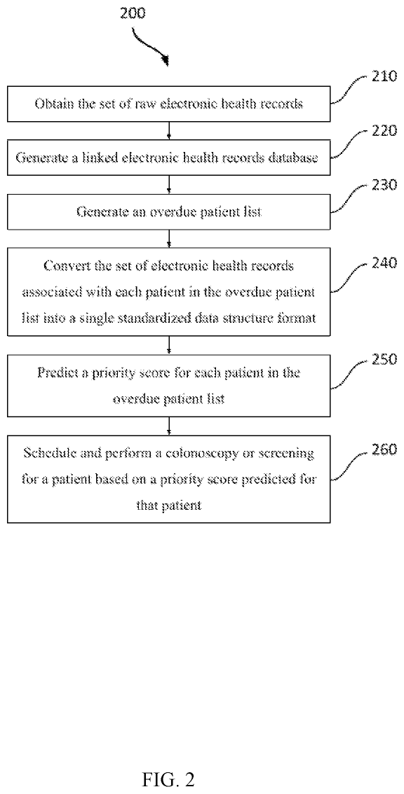

FIG. 2 illustrates a method 200 for facilitating timely prophylactic colorectal cancer evaluations based on aggregated electronic health records, in accordance with one embodiment of the present invention.

Firstly, if required, a healthcare provider 54 employs the system to convert their electronic health records 52 into one or more data formats. This conversion is compatible with the formats that the recommended recall model 40, the feature extraction model 42, and the metadata extractor 46 are designed to handle. In the preferred embodiment, the healthcare provider can use an electronic device 56 as an access point to facilitate this process.

At step 210, the healthcare administrator begins by selecting one or more providers for whom to trigger analysis using the interface displayed on the electronic device, thereby defining a pool of patients 58 whose data will be analyzed. In other embodiments, the healthcare administrator begins by selecting one or more patients whose data will be analyzed from the pool of patients seen and treated by the administrator's hospital or clinic. Next, at step 220, the electronic health records 52 relating to all patients for the selected providers or relating to all selected patients are processed and organized by the Data Linker 48 to generate a Linked Electronic Health Record Database 50.

The OPLG 62 then uses the linked database 50 to identify a singular patient's relevant information (e.g., procedure reports, clinical notes, pathology reports) from the stored processed data 28 associated with the selected pool of patients 58. The clinical notes and procedure reports from the identified information are then processed by the RRM 40 to generate a recommended recall interval. The individual patient's procedure reports, clinical notes, and pathology reports are processed by the metadata extractor 46 to generate various data such as the time and date of the reports/notes as well as the last documented procedure date.

The OPLG 62 then compares the recommended recall interval generated by the RRM 40 to the last documented procedure date identified by the metadata extractor 46. Here, the recommended recall interval corresponding to the most recent procedure date as identified by the metadata extractor 46 defines a window from that last documented procedure date during which the patient should have completed a follow-up colonoscopy or screening. Patients for whom that time window has fully elapsed without receiving a follow-up colonoscopy or screening are designated as “overdue” and are added to the overdue patients list 64 at step 230.

Then, for each patient on the overdue patients list, the standard format converter 66 converts their related electronic health records from the linked database 50 into a standardized data structure format 68 at step 240. The priority score model 70 then uses said electronic health records in a standardized data structure format 68 as input to generate a recall priority score 72 between 1 and 10 for each patient on the overdue patients list at step 250. The overdue patients list and the patient's respective predicted recall priority score are displayed to the healthcare provider via a display interface of the electronic device 56. Optionally, relevant past medical events may be displayed.

In practice, the healthcare administrator and provider then work together, using the displayed recall priority scores, to decide which patients should receive prophylactic colonoscopies and screenings. For instance, and without limitation, a recall priority score above 7 would typically indicate a need for an urgent and comprehensive colonoscopy or screening/surveillance exam within the next three months. This threshold of 7 is set to identify approximately the top 20th percentile of patients by colorectal cancer risk. If hospital resource limitations change, this threshold can be easily adjusted by the healthcare provider as needed.

The healthcare provider then contacts patients with a recall priority score over the set cutoff, schedules a prophylactic colonoscopy or screening/surveillance exam for said patients within the next three months, and performs said scheduled prophylactic colonoscopy or screening/surveillance exam for said patients at step 260. As one skilled in the art will appreciate, these steps may, but do not necessarily have to, be performed by the same individual. For example, a nurse may use the system to determine which patients require urgent follow-up procedures, an administrative assistant may contact said patients and schedule said follow-up procedures, and a doctor may perform said follow-up procedures for said patients.

It will be appreciated that while FIG. 1 shows the receipt of input electronic health records from one or more patients from a single hospital or clinic's pool of patients, in practice this may be occurring essentially simultaneously for other many other patients across one or more medical systems, hospitals, or clinics, depending on the extent of the roll-out of the system. The system preferably employs sufficient computing resources for the computer 38 (or system of computers) to operate the models on the input health records and generate data as to predictions for colorectal cancer and colorectal cancer risks for all of said patients' electronic health records simultaneously in real-time and transmit the data to the electronic device(s) 56 for display on the healthcare provider-facing interface of the device.

A partial summary of our findings from development and testing of the models is as follows. The disclosed system was implemented and tested over a three-month period at a large, tertiary care center. During this test period, 4663 missed recalls for patients were recorded, with 667 of those patients being identified as high risk (high risk being defined as patients due for a recall within 2 years). Out of those 667 high risk patients, 162 were identified by the system as being overdue, including a subset of patients with high-risk indications such as a personal history of colon cancer. Among the 162 patients identified as overdue for their appointments, a manual review affirmed that 96% of these identifications were accurate. In other words, the vast majority of patients flagged by the system as overdue were indeed overdue according to the manual verification. Following the recommendations of the implemented system, subsequent outreach to this high risk group based were performed, with a total of 33 precancerous lesions from 17 patients identified and removed on subsequent colonoscopy (of a total of 44 patients evaluated).

What is claimed is:

1. A system, comprising in combination:
a) computer memory storing aggregated electronic health records from a multitude of patients of diverse age, health conditions, and demographics including as elements thereof at least size and history of polyps and cancers identified during previous screenings, laboratory values, vital signs, and medical notes and obtained in different formats, wherein the aggregated electronic health records are converted into a single standardized data structure format and ordered per patient into an ordered arrangement; and
b) a computer executing one or more deep learning models to predict a recall priority score based on an input electronic health record of a patient in a standardized data structure format, and wherein the computer executing the one or more deep learning models comprises:
(1) generating, for a plurality of electronic health records, a linked electronic health records database that joins pointers to individual documents from the electronic health records that correspond to the same visit for each patient from the multitude of patients based on metadata;
(2) generating an overdue patient list containing patients overdue for a colonoscopy or screening from the multitude of patients represented in the linked electronic health records database;
(3) converting, for each patient in the overdue patient list, the set of electronic health records associated with each patient into a single standardized data structure format; and,
(4) predicting, for each patient in the overdue patient list, a priority score based on each patient's electronic health records in the standardized data structure format.

2. The system of claim 1, further comprising an electronic device equipped with a screen display configured with a healthcare provider-facing interface.

3. The system of claim 2, wherein the metadata is extracted from the plurality of elements of the electronic health records.

4. The system of claim 3, wherein the generating an overdue patient list comprises:
generating, for each patient from the multitude of patients represented in the linked electronic health records database, a last documented procedure date based on the extracted document metadata; and,
generating, for each patient from the multitude of patients represented in the linked electronic health records database, a recommended recall interval based on the patient's documents in the linked health records database;
wherein patients are added to the overdue patients list based on the recommended recall interval and the last document procedure date for each patient.

5. A method of facilitating timely prophylactic colorectal cancer evaluations based on aggregated electronic health records having a plurality of elements from a multitude of patients, the method comprising:
obtaining the set of raw electronic health records;
generating, for a plurality of electronic health records, a linked electronic health records database that joins pointers to individual documents from the electronic health records that correspond to the same visit for each patient from the multitude of patients based on the extracted metadata;
generating an overdue patient list containing patients overdue for a colonoscopy or screening from the multitude of patients represented in the linked electronic health records database;
converting, for each patient in the overdue patient list, the set of electronic health records associated with each patient into a single standardized data structure format; and,
predicting, for each patient in the overdue patient list, a priority score based on each patient's electronic health records in the standardized data structure format.

6. The method of claim 5, wherein the metadata is extracted from the plurality of elements of the electronic health records.

7. The method of claim 6, wherein the generating an overdue patient list comprises:
generating, for each patient from the multitude of patients represented in the linked electronic health records database, a last documented procedure date based on the extracted document metadata; and,
generating, for each patient from the multitude of patients represented in the linked electronic health records database, a recommended recall interval based on the patient's documents in the linked health records database;
wherein patients are added to the overdue patients list based on the recommended recall interval and the last document procedure date for each patient.

8. The method of claim 7, further comprising the step of a medical provider scheduling and performing a colonoscopy or screening for a patient based on a priority score predicted for that patient.

9. A system, comprising in combination:
computer memory storing aggregated electronic health records from a multitude of patients of diverse age, health conditions, and demographics including as elements thereof at least size and history of polyps and cancers identified during previous screenings, laboratory values, vital signs, and medical notes and obtained in different formats, wherein the aggregated electronic health records are converted into a single standardized data structure format and ordered per patient into an ordered arrangement; and,
a computer executing one or more deep learning models to predict a recall priority score based on an input electronic health record of a patient in a standardized data structure format, and wherein the computer executing the one or more deep learning models comprises:
(1) generating textual data for electronic health records not in a digital format from the plurality of electronic health records so the entirety of the electronic health records are in a digital format;
(2) extracting metadata from the plurality of elements of the electronic health records;
(3) generating, for a plurality of electronic health records, a linked electronic health records database that joins pointers to individual documents from the electronic health records that correspond to the same visit for each patient from the multitude of patients based on metadata;
(4) generating, for each patient from the multitude of patients represented in the linked electronic health records database, a last documented procedure date based on the extracted document metadata;
(5) generating, for each patient from the multitude of patients represented in the linked electronic health records database, a recommended recall interval based on the patient's documents in the linked health records database;
(6) generating an overdue patient list containing patients overdue for a colonoscopy or screening from the multitude of patients represented in the linked electronic health records database wherein patients are added to the overdue patients list based on the recommended recall interval and the last document procedure date for each patient;
(7) converting, for each patient in the overdue patient list, the set of electronic health records associated with each patient into a single standardized data structure format; and,
(8) predicting, for each patient in the overdue patient list, a priority score based on each patient's electronic health records in the standardized data structure format.

10. The system of claim 9, further comprising an electronic device equipped with a screen display configured with a healthcare provider-facing interface.
